(19) United States
(12) Patent Application Publication
(10) Pub. No.: US 2002/0182192 A1
(43) Pub. Date: Dec. 5, 2002

(54) Methods for treating muscular dystrophy

(75) Inventors: Louis M. Kunkel, Westwood, MA (US); Emanuela Gussoni, Winchester, MA (US); Richard C. Mulligan, Lincoln, MA (US); Yuko Soneoka, Washington, DC (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Children's Medical Center Corporation, Boston, MA (US)

(21) Appl. No.: 10/100,268

(22) Filed: Mar. 14, 2002

Related U.S. Application Data
(63) Continuation of application No. PCT/US00/25128, filed on Sep. 14, 2000.
(60) Provisional application No. 60/153,821, filed on Sep. 14, 1999.

(51) Int. Cl. A61K 48/00; C12N 5/08

(52) U.S. Cl. 424/93.21; 424/93.7; 435/372

(57) ABSTRACT

Methods for treating muscle diseases via bone marrow transplantation of either allogeneic cells or autologous cells engineered to express dystrophin or other gene products affected in muscle diseases are disclosed. Bone marrow cells and bone marrow SP cells (a highly purified population of hematopoietic stem cells) can be used in the methods. Muscle diseases include muscular dystrophies, such as Duchenne muscular dystrophy, Becker muscular dystrophy and limb-girdle muscular dystrophies.

### RELATED APPLICATIONS

This application is a continuation of International Application No. PCT/US00/25128, filed Sep. 14, 2000, which claims the benefit of U.S. Provisional Application No. 60/153,821, filed Sep. 14, 1999. The teachings of the above applications are incorporated herein by reference in their entirety.

### GOVERNMENT SUPPORT

The invention was supported, in whole or in part, by Grant No. HL54785 from the National Institutes of Health. The United States Government has certain rights in the invention.

### BACKGROUND OF THE INVENTION

There are probably about 3,000 muscle proteins, each encoded by a different gene. Some muscle proteins are part of the structure of muscle fibers, while others influence chemical reactions in the fibers. A defect in a muscle protein gene can lead to a muscle disease. The precise defect in a muscle protein gene can influence the nature and severity of a muscle disease.

Muscular dystrophies are caused by defects in muscle protein genes and are typically progressive disorders mainly of striated muscle that lead to breakdown of muscle integrity, often resulting in death. The histologic picture shows variation in fiber size, muscle cell necrosis and regeneration, and often proliferation of connective and adipose tissue. The precise defect in a muscle protein gene determines the nature and severity of a muscular dystrophy. For example, two major types of muscular dystrophy, Duchenne muscular dystrophy (DMD) and Becker muscular dystrophy (BMD), are allelic, lethal degenerative muscle diseases. DMD results from mutations in the dystrophin gene on the X-chromosome (Hoffinan et al., N. Engl. J. Med., 318:1363-1368 (1988)), which usually result in the absence of dystrophin, a cytoskeletal protein in skeletal and cardiac muscle. BMD is the result of mutations in the same gene (Hoffinan et al., N. Engl. J. Med., 318:1363-1368 (1988)), but dystrophin is usually expressed in muscle but at a reduced level and/or as a shorter, internally deleted form, resulting in a milder phenotype.

Previous attempts to restore dystrophin expression in X-linked muscular dystrophic (mdx) mice either by cell transplantation (Karpati, G. et al., Am. J. Pathol., 135:27-32 (1989); Morgan, J. E. et al., J. Cell Biol., 111:2437-2449 (1990); and Partridge, T. A. et al., 337:176-179 (1989)) or by in vivo gene transfer (Acsadi, G. et al., Nature, 352:815-818 (1991); Ragot, T. et al., Nature, 361:647-650 (1993); Vincent, N. et al., Nat. Genet., 5:130-134 (1993); and Kochanek, S. et al., Proc. Natl. Acad Sci. USA, 93:5731-5736 (1996)) have been quite successful but not surprisingly, have resulted only in the restoration of dystrophin expression close to the site of injection of cells or vector (Partridge, T. A., Muscle Nerve, 14:197-212 (1991)).

The ability to restore dystrophin expression, as well as the expression of other gene products affected in a particular muscle disease, beyond the site of injection of cells or vectors, would have important therapeutic application for treatment of muscle diseases. Thus, there is a considerable need in developing alternative approaches for restoring expression of gene products affected in muscle diseases in mammals.

### SUMMARY OF THE INVENTION

The present invention provides methods for treating a muscle disease in a mammal (e.g. a human or other mammal or vertebrate) in need thereof via bone marrow transplantation. In one embodiment, the method comprises administering an effective amount of bone marrow cells to the mammal. In a second embodiment, the method comprises administering an effective amount of bone marrow SP cells (a highly purified population of hematopoietic stem cells) to the mammal. In a third embodiment, the method comprises (a) introducing a nucleic acid sequence of interest encoding a desired nucleic acid product into bone marrow cells from the mammal; and (b) administering the cells produced in step (a) to the mammal. In a fourth embodiment, the method comprises (a) introducing a nucleic acid sequence of interest encoding a desired nucleic acid product into bone marrow SP cells obtained from the mammal; and (b) administering the cells produced in step (a) to the mammal. In a fifth embodiment, the method comprises (a) introducing a nucleic acid sequence of interest encoding a desired nucleic acid product into donor bone marrow cells; and (b) administering the cells produced in step (a) to the mammal. In a sixth embodiment, the method comprises (a) introducing a nucleic acid sequence of interest encoding a desired nucleic acid product into donor bone marrow SP cells; and (b) administering the cells produced in step (a) to the mammal.

The present invention also provides uses of bone marrow cells and bone marrow SP cells for the manufacture of medicaments for use in the treatment of a muscle disease in a mammal. In a particular embodiment, a nucleic acid sequence of interest encoding a desired nucleic acid product is introduced into the bone marrow cells or bone marrow SP cells.

The present invention further relates to bone marrow cells and bone marrow SP cells that are used in the methods and uses described herein.

A desired nucleic acid product, as used herein, refers to the desired protein or polypetide, DNA or RNA (e.g., gene product) to be expressed in a mammal in treatment of the mammal's muscle disease. The desired nucleic acid product is otherwise defective or absent in the mammal. In a particular embodiment, the desired nucleic acid product is dystrophin. Other desired nucleic acid products include calpain-3, sarcoglycan complex members (e.g., α-sarcoglycan, β-sarcoglycan, γ-sarcoglycan and δ-sarcoglycan) and laminin α2-chain.

Generally, a nucleic acid sequence encoding a desired nucleic acid product will be introduced into bone marrow cells or bone marrow SP cells through the use of viral vectors, such as DNA or RNA (retroviral) vectors. Retroviruses have been shown to have properties which make them particularly well suited to serve as recombinant vectors by which a nucleic acid sequence encoding a desired nucleic acid product can be introduced into mammalian (e.g., human or other mammal or vertebrate) cells. For example, recombinant retrovirus for use in delivery of a desired nucleic acid product can be generated by introducing a suitable proviral DNA vector encoding the desired nucleic acid product into fibroblastic cells that produce the viral proteins necessary for encapsidation of the desired recombinant RNA. This is one approach which can be used to introduce (deliver) a nucleic acid sequence encoding a desired nucleic acid product into bone marrow cells or bone marrow SP cells for the purpose of treating a muscle disease in a mammal. See, for example, Mann, R. et al., Cell, 33:153-159 (1983); Watanabe, S. and H. M. Temin, Mol. Cell. Biol., 3:2241-2249 (1983); Cone, R. D. and R. C. Mulligan, Proc. Natl. Acad. Sci. USA, 81:6349-6353 (1984); Soneoka, Y. et al., Nucl. Acids Research, 123:628-633 (1995); and Danos, O. and R. C. Mulligan, U.S. Pat. No. 5,449,614.

Muscle diseases which can be treated using the methods of the present invention include muscular dystrophies. Muscular dystrophies which can be treated using the methods of the present invention include Duchenne muscular dystrophy (DMD) Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies and Emery-Dreifuss muscular dystrophy.

### DETAILED DESCRIPTION OF THE INVENTION

The results described herein show that after transplantation of either normal (unfractionated) bone marrow cells or a highly purified population of hematopoietic stem cells, termed bone marrow SP cells, from normal donors into X-linked muscular dystrophic (mdx) recipients, dystrophin-negative myofibers can become converted to dystrophin-positive myofibers. In both cases, dystrophin expression was restored in about 1-10% of the myofibers (Tables 1 and 2). In one animal, dystrophin expression was evident in close to 10% of all myofibers (Table 1), a level of restoration thought by some to be potentially useful clinically (Phelps, S. F. et al., Hum. Mol. Genet., 4:1251-1258 (1995)). Donor-derived nuclei were also found in both cases to be both centrally and peripherally located within myofibers, indicating the restoration of dystrophin in both regenerating and mature myofibers (Table 3). Overall, these results suggest that the delivery, via bone marrow transplantation, of either allogeneic cells or autologous cells engineered to express dystrophin or the gene products affected in other related muscle diseases can provide a new avenue for treating muscular dystrophy and other devastating muscle diseases.

As a result, the present invention provides methods for treating a muscle disease in a mammal (e.g. a human or other mammal or vertebrate) in need thereof via bone marrow transplantation. Such as a mammal is also referred to herein as a recipient. In one embodiment, the method comprises administering an effective amount of donor bone marrow cells to the mammal. In a second embodiment, the method comprises administering an effective amount of donor bone marrow SP cells (a highly purified population of hematopoietic stem cells) to the mammal. In a third embodiment, the method comprises (a) introducing a nucleic acid sequence of interest encoding a desired nucleic acid product into bone marrow cells from the mammal; and (b) returning the cells produced in step (a) to the mammal. In a fourth embodiment, the method comprises (a) introducing a nucleic acid sequence of interest encoding a desired nucleic acid product into bone marrow SP cells obtained from the mammal; and (b) returning the cells produced in step (a) to the mammal. In a fifth embodiment, the method comprises (a) introducing a nucleic acid sequence of interest encoding a desired nucleic acid product into donor bone marrow cells; and (b) administering the cells produced in step (a) to the mammal. In a sixth embodiment, the method comprises (a) introducing a nucleic acid sequence of interest encoding a desired nucleic acid product into donor bone marrow SP cells; and (b) administering the cells produced in step (a) to the mammal.

As used herein, muscle diseases include, but are not limited to, recessive or inherited myopathies, such as, but not limited to, muscular dystrophies. Muscular dystrophies are genetic diseases characterized by progressive weakness and degeneration of the skeletal or voluntary muscles which control movement. The muscles of the heart and some other involuntary muscles are also affected in some forms of muscular dystrophy. Muscular dystrophies are described in the art and include Duchenne muscular dystrophy (DMD), Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies and Emery-Dreifuss muscular dystrophy. See, e.g., Hoffinan et al., N. Engl. J. Med., 318:1363-1368 (1988); Bönnemann, C. G. et al., Curr. Opin. Ped., 8:569-582 (1996); Worton, R., Science, 270:755-756 (1995); Funakoshi, M. et al., Neuromuscul. Disord., 9(2):108-114 (1999); Lim, L. E. and Campbell, K. P., Curr. Opin. Neurol., 11(5):443-452 (1998); Voit, T., Brain Dev., 20(2):65-74 (1998); Brown, R. H., Annu. Rev. Med., 48:457-466 (1997); Fisher, J. and Upadhyaya, M., Neuromuscul. Disord., 7(1):55-62 (1997), which references are incorporated entirely incorporated herein by reference.

Bone marrow cells can be obtained from any mammal according to methods generally known in the art. For example, bone marrow can be obtained from a donor by inserting needles into the marrow cavity and aspiration of the marrow. Marrow is passed through screens to separate the bone marrow cells.

Bone marrow SP cells and methods of obtaining bone marrow SP cells from any mammal are described in Goodell, M. A. et al., J. Exp. Med., 183:1797-1806 (1996); and Goodell, M. A. et al., International Publication No. WO 9639489 (published Dec. 12, 1996), which references are entirely incorporated herein by reference. Bone marrow SP cells are also referred to herein as a highly purified population of hematopoietic stem cells.

Bone marrow cells and bone marrow SP cells used in the methods of the present invention must be living (e.g., nucleated). The cells used can be obtained from a mammal to whom they will be returned or from another/different mammal of the same or different species (donor) and introduced into a recipient. For example, the cells can be obtained from a pig and introduced into a human.

The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminents (e.g., cows, pigs, horses).

As used herein, “donor” refers to a mammal that is the natural source of the bone marrow cells or bone marrow SP cells. The donor can be a healthy mammal (i.e., a mammal that is not suffering from a muscle disease). Alternatively, the donor is a mammal with a muscle disease. A recipient is a mammal suffering from a muscle disease. In a particular embodiment, the recipient is a mammal with a muscular dystrophy. In an embodiment of particular interest, the recipient is a human patient.

In a particular embodiment, the donor and recipient are matched for immunocompatibility. Preferably, the donor and the recipient are matched for their compatibility for the major histocompatibility complex (MHC) (human leukocyte antigen (HLA))-class I (e.g., loci A,B,C) and -class II (e.g., loci DR, DQ, DRW) antigens. Immunocompatibility between donor and recipient are determined according to methods generally known in the art (see, e.g., Charron, D. J., Curr. Opin. Hematol., 3:416-422 (1996); Goldman, J., Curr. Opin. Hematol., 5:417-418 (1998); and Boisjoly, H. M. et al., Opthalmology, 93:1290-1297 (1986)).

Nucleic acid sequences are defined herein as heteropolymers of nucleic acid molecules. The nucleic acid molecules can be double stranded or single stranded and can be a deoxyribonucleotide (DNA) molecule, such as cDNA or genomic DNA, or a ribonucleotide (RNA) molecule. As such, the nucleic acid sequence can, for example, include one or more exons, with or without, as appropriate, introns, as well as one or more suitable control sequences. In one example, the nucleic acid molecule contains a single open reading frame which encodes a desired nucleic acid product. The nucleic acid sequence is operably linked to a suitable promoter.

A nucleic acid sequence encoding a desired nucleic acid product can be isolated from nature, modified from native sequences or manufactured de novo, as described in, for example, Ausubel et al., Current Protocols in Molecular Biology, John Wiley & Sons, New York (1998); and Sambrook et al., Molecular Cloning: A Laboratory Manual, 2nd edition, Cold Spring Harbor University Press, New York. (1989). Nucleic acids can be isolated and fused together by methods known in the art, such as exploiting and manufacturing compatible cloning or restriction sites.

Typically, the nucleic acid sequence will be a gene which encodes the desired nucleic acid product. In this case, the desired nucleic acid product is a gene product. Such a gene is typically operably linked to suitable control sequences capable of effecting the expression of the desired nucleic acid product in bone marrow cells and bone marrow SP cells. The term “operably linked”, as used herein, is defined to mean that the gene (or the nucleic acid sequence) is linked to control sequences in a manner which allows expression of the gene (or the nucleic acid sequence). Generally, operably linked means contiguous.

Control sequences include a transcriptional promoter, an optional operator sequence to control transcription, a sequence encoding suitable mRNA ribosomal binding sites and sequences which control termination of transcription and translation. In a particular embodiment, a recombinant gene (or a nucleic acid sequence) encoding a desired nucleic acid product can be placed under the regulatory control of a promoter which can be induced or repressed, thereby offering a greater degree of control with respect to the level of the product in the bone marrow cells or bone marrow SP cells.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known in the art. Exemplary promoters include the SV40 and human elongation factor (EFI). Other suitable promoters are readily available in the art (see, e.g., Ausubel et al., Current Protocols in Molecular Biology, John Wiley & Sons, Inc., New York (1998); Sambrook et al., Molecular Cloning: A Laboratory Manual, 2nd edition, Cold Spring Harbor University Press, New York (1989); and U.S. Pat. No. 5,681,735).

A desired nucleic acid product, as used herein, refers to the desired protein or polypetide, DNA or RNA (e.g., gene product) to be expressed in a mammal in treatment of the mammal's muscle disease. The desired nucleic acid product is otherwise defective or absent in the mammal. In a particular embodiment, the desired nucleic acid product is a gene product affected in a particular muscle disease. For example, in the treatment of a mammal with DMD or BMD, the desired nucleic acid product can be dystrophin. In the treatment of a mammal with a limb-girdle muscular dystrophy, desired nucleic acid products include, but are not limited to, calpain-3 and sarcoglycan complex members (e.g., α-sarcoglycan, β-sarcoglycan, γ-sarcoglycan and δ-sarcoglycan). In the treatment of a mammal with a congenital muscular dystrophy, desired nucleic acid products include, but are not limited to, laminin α2-chain.

Nucleic acid sequences encoding a desired nucleic acid product can be introduced into bone marrow cells or bone marrow SP cells by a variety of methods (e.g., transfection, infection, transformation, direct uptake, projectile bombardment, using liposomes). In a particular embodiment, a nucleic acid sequence encoding a desired nucleic acid product is inserted into a nucleic acid vector, e.g., a DNA plasmid, virus or other suitable replicon (e.g., viral vector). As a particular example, a nucleic acid sequence encoding a desired nucleic acid product is integrated into the genome of a virus which is subsequently introduced into purified bone marrow cells or bone marrow SP cells. The term “integrated”, as used herein, refers to the insertion of a nucleic acid sequence (e.g., a DNA or RNA sequence) into the genome of a virus as a region which is covalently linked on either side to the native sequences of the virus. Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

Packaging cell lines can be used for generating recombinant viral vectors comprising a recombinant genome which includes a nucleotide sequence (RNA or DNA) encoding a desired nucleic acid product. The use of packaging cell lines can increase both the efficiency and the spectrum of infectivity of the produced recombinant virons.

Packaging cell lines useful for generating recombinant viral vectors comprising a recombinant genome which includes a nucleotide sequence encoding a desired nucleic acid product are produced by transfecting host cells, such as mammalian host cells, with a viral vector including the nucleic acid sequence encoding the desired nucleic acid product integrated into the genome of the virus, as described herein. Suitable host cells for generating cell lines include human (such as HeLa cells), bovine, ovine, porcine, murine (such as embryonic stem cells), rabbit and monkey (such as COS 1 cells) cells. A suitable host cell for generating a cell line may be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions). These cells can be obtained commercially or from a depository or obtained directly from an individual, such as by biopsy. Viral stocks are harvested according to methods generally known in the art. See, e.g., Ausubel et al., Eds., Current Protocols In Molecular Biology, John Wiley & Sons, New York (1998); Sambrook et al., Eds., Molecular Cloning: A Laboratory Manual, 2nd edition, Cold Spring Harbor University Press, New York (1989); Danos and Mulligan, U.S. Pat. No. 5,449,614; and Mulligan and Wilson, U.S. Pat. No. 5,460,959, the teachings of which are incorporated herein by reference.

Examples of suitable methods of transfecting or transforming cells include infection, calcium phosphate precipitation, electroporation, microinjection, lipofection and direct uptake. Such methods are described in more detail, for example, in Sambrook et al., Molecular Cloning: A Laboratory Manual, Second Edition, Cold Spring Harbor University Press, New York (1989); Ausubel, et al., Current Protocols in Molecular Biology, John Wiley & Sons, New York (1998); and Danos and Mulligan, U.S. Pat. No. 5,449,614, the teachings of which are incorporated herein by reference.

Virus stocks consisting of recombinant viral vectors comprising a recombinant genome which includes a nucleotide (DNA or RNA) sequence encoding a desired nucleic acid product, are produced by maintaining the transfected cells under conditions suitable for virus production (e.g., in an appropriate growth media and for an appropriate period of time). Such conditions, which are not critical to the invention, are generally known in the art. See, e.g., Sambrook et al., Molecular Cloning: A Laboratory Manual, Second Edition, Cold Spring Harbor University Press, New York (1989); Ausubel et al., Current Protocols in Molecular Biology, John Wiley & Sons, New York (1998); U.S. Pat. Nos. 5,449,614; and 5,460,959, the teachings of which are incorporated herein by reference.

A vector comprising a nucleic acid sequence encoding a desired nucleic acid product can also be introduced into bone marrow cells or bone marrow SP cells by targeting the vector to cell membrane phospholipids. For example, targeting of a vector can be accomplished by linking the vector molecule to a VSV-G protein, a viral protein with affinity for all cell membrane phospholipids. Such a construct can be produced using methods well known to those practiced in the art.

As a particular example of the above approach, a recombinant gene (or a nucleic acid sequence) encoding a desired nucleic acid product and which is operably linked to control sequences capable of effecting the expression of the desired nucleic acid product in bone marrow cells or bone marrow SP cells can be integrated into the genome of a virus that enters the particular cells of interest. By infection, the bone marrow cells or bone marrow SP cells can be genetically altered to comprise a stablely incorporated recombinant gene (or a nucleic acid sequence) encoding a desired nucleic acid product. Bone marrow cells and bone marrow SP cells genetically altered in this way (recombinant bone marrow cells and recombinant bone marrow SP cells) can then be examined for expression of the recombinant gene (or nucleic acid sequence) prior to administration to a mammal (recipient). For example, the amount of desired nucleic acid product expressed can be measured according to standard methods (e.g., by immunoprecipitation). In this manner, it can be determined in vitro whether a desired nucleic acid product is capable of expression to a suitable level (desired amount) in the bone marrow cells or bone marrow SP cells prior to administration to a mammal. Genetically altered bone marrow cells (recombinant bone marrow cells) and genetically altered bone marrow SP cells (recombinant bone marrow SP cells) expressing the desired nucleic acid product to a suitable level can be expanded (grown) for introduction (infusion) into the mammal. Methods for expanding (growing) cells are well known in the art.

Bone marrow cells and bone marrow SP cells, either genetically altered as described herein or unaltered, can be administered to (transplanted, implanted, introduced into) a mammal according to methods known to those practiced. For example, bone marrow transplantation (BMT) has been described for a number of diseases involving hematopoietic cells, including leukemia (Gorin, N. C. et al., Br. J. Haematol, 64:385-395 (1986), Ringden, O. et al., Leuk Lymphoma, 24:71-79 (1996)), aplastic anemia (O'Reilly, R. J., Blood, 62:941-964 (1983); and Gordon-Smith, E. C., Hematol. Oncol., 5:255-263 (1987)), thalassemia major (Thomas, E. D., Prog. Clin. Biol. Res., 309:187-191 (1989); and Lucarelli, G. et al., Exp. Hematol., 12:676-681 (1984)), and the immunodeficiencies (Kenny, A. B. and Hitzig, W. H., Eur. J. Pediatr., 131:155-177 (1979); and Parkman, R. et al., N. Engl. J. Med., 298:921-927 (1978)). Preferably, the mode of administration is intravenously, including infusion and/or bolus injection, or intraperitoneally by injection. Other modes of administration (parenteral, mucosal, implant, intramuscular, intradermal, transdermal (e.g., in slow release polymers), are generally known in the art. Preferably, the cells are administered in a medium suitable for the particular mode and route of administration into a mammal, such as phosphate buffered saline.

An “effective amount” of bone marrow cells or bone marrow SP cells, either genetically altered as described herein or unaltered, is defined herein as that amount of bone marrow cells which, when administered to a mammal, is sufficient for therapeutic efficacy (e.g., an amount sufficient for significantly reducing or eliminating symptoms and/or signs associated with the disease). The amount of bone marrow cells or bone marrow SP cells, either genetically altered as described herein or unaltered, administered to a mammal, including frequency of administration, will vary depending upon a variety of factors, including mode and route of administration; size, age, sex, health, body weight and diet of the recipient; nature and extent of symptoms of the muscle disease being treated; kind of concurrent treatment, frequency of treatment, and the effect desired.

Many of the parameters which affect the extent of restoration of dystrophin by bone marrow-derived cells, and the temporal dynamics of the reconstitution process are not known. In light of the fact that very small numbers of bone marrow SP cells are clearly capable of giving rise to the rather large numbers of dystrophin-positive myofibers, and that there is a significant lag in time between the injection of the cells and emergence of detectable dystrophin-positive fibers, it is likely that the transplanted cells must colonize and expand within the microenvironment of the bone marrow, as normally occurs after BMT. The optimal preparation of cells for transplantation, and the nature of the requirements, if any, for conditioning the recipient so as to achieve efficient engraftment of dystrophin positive cells may be determined. Such determination can be made by damaging or injuring the recipient muscle prior to injection, for example. If reconstitution of the bone marrow compartment is a prerequisite for efficient contribution of the cells to muscle, then bone marrow-derived hematopoietic stem cells may prove to represent a unique source of precursor cells for the engraftment of muscle.

It is noteworthy that in all of the experiments reported herein, not all dystrophin positive myofibers could be associated with Y-positive (donor) nuclei. This may be due to a number of factors. First, it has been observed that the hybridization efficiency of the Y chromosome probe to male-derived tissue sections is 70-80%, compared to over 90% on lymphocytes, and therefore it was expected that not all Y-positive nuclei would be detected by the analysis described herein (Gussoni, E. et al., Nat. Biotechnol., 14:1012-1016 (1996)). Furthermore, studies on nuclear domains in myotubes have shown that a single nucleus can produce a membrane-associated protein that becomes expressed along as much as 180 μm of a muscle fiber (Gussoni, E. et al., Nature Medicine, 3:970-977 (1997); Hall, Z. W. and Ralston, E., Cell, 59:771-772 (1989); Pavlath, G. K. et al., Nature, 337:570-573 (1989); and Ralston, E. and Hall, Z. W., J. Cell Biol., 109:2345-2352 (1989)). Indeed, in the studies described herein, dystrophin-positive fibers extending up to approximately 1 mm in length were observed, making them more difficult to search for the donor nuclei that contribute to dystrophin expression. Lastly, it is possible that some proportion of the dystrophin positive fibers which do not contain Y-positive nuclei may result from revertant dystrophin expression, as has been reported previously (Hoffinan, E. P. et al., J. Neurol. Sci., 99:9-25 (1990)).

As discussed above, previous attempts to restore dystrophin expression in the mdx mice either by muscle cell transplantation (Karpati, G. et al., Am. J. Pathol., 135:27-32 (1989); Morgan, J. E. et al., J. Cell Biol., 111:2437-2449 (1990); and Partridge, T. A. et al., 337:176-179 (1989)) or by in vivo gene transfer (Acsadi, G. et al., Nature, 352:815-818 (1991); Ragot, T. et al., Nature, 361:647-650 (1993); Vincent, N. et al., Nat. Genet., 5:130-134 (1993); and Kochanek, S. et al., Proc. Natl. Acad Sci. USA, 93:5731-5736 (1996)) have been quite successful but not surprisingly, have resulted only in the restoration of dystrophin expression close to the site of injection of cells or vector (Partridge, T. A., Muscle Nerve, 14:197-212 (1991)). It is clear from the results presented herein that the transplantation of bone marrow and/or HSCs offers the unique possibility of systemic delivery of therapeutic cells via the circulation and thereby the improved distribution of dystrophin-positive cells to the overall muscle mass of an individual.

The requirement for the use of only very small numbers of purified hematopoietic stem cells for transplantation would greatly facilitate therapeutic strategies requiring gene transfer or gene repair. Another potential advantage of the use of hematopoietic stem cells for cell therapy and /or gene therapy is that the use of stem cells may result in the continued production of myogenic precursors over the lifetime of an individual, as is achieved for blood-derived cells. If new myogenic cells are incorporated over time, there could be progressive improvements in muscle function. It is interesting, in this regard, that in the studies described herein, the proportion of myofibers demonstrating dystrophin expression appeared to increase at longer times after transplantation.

Finally, the studies with bone marrow-derived SP cells described herein provide the first direct evidence that hematopoietic stem cells with the capacity for the complete reconstitution of lethally irradiated recipients have the potential to differentiate into muscle. The studies described herein also raise the intriguing possibility that hematopoietic stem cells normally provide a means of repairing and replenishing a variety of cells and tissues of non-hematopoietic origin. If so, BMT may ultimately prove to be applicable to cell and ex vivo gene therapies involving a range of different cell types.

The present invention will now be illustrated by the following examples, which are not to be considered limiting in any way.

### EXAMPLES

The following methods were used in Examples 1, 2 and 3.

### Animals

The dystrophin-deficient mdx mouse used in the experiments described herein is an animal model of Duchenne's muscular dystrophy (DMD) (Sicinski, P. et al., Science, 244:1578-1580 (1989); and Bulfield, G. et al., Proc. Natl. Acad. Sci. USA, 81:1189-1192 (1984)) and serves as a good approximation to human disease. The mdx mouse has the same genetic defect as occurs in DMD. As in DMD, its muscle fibers lack the protein dystrophin (Hoffinan, E. P. et al., Cell, 51:919-928 (1987)) and undergo widespread degeneration.

Mice were obtained at 6-8 weeks of age and were used between 6 and 12 weeks of age. C57BL/10 mice and C57BL/10ScSn-Dmd^{mdx}/J (Z-linked muscular dystrophy) mice were both obtained from The Jackson Laboratory (Bar Harbor, Me.).

### Purification of Bone Marrow and Hematopoietic Stem Cells

Bone marrow was extracted from the femurs and tibias and red blood cells were removed with ammonium chloride solution (Sigma). For the purification of hematopoietic stem cells, bone marrow cells were resuspended at 106 cells/ml in HBSS containing 2% fetal calf serum (FCS), 1 mM HEPES, and 5 μg/ml Hoechst 33342 (Sigma) and incubated at 37° C. for 90 minutes as previously described (Goodell, M. A. et al., J. Exp. Med., 183:1797-1806 (1996); and Goodell, M. A. et al., International Publication No. WO 9639489 (published Dec. 12, 1996), the teachings of which are entirely incorporated herein by reference).

After staining, cells were magnetically preenriched for Sca-1-positive cells using the MACS (Miltenyi Biotec) and streptavidin microbeads. Prior to cell sorting, cells were resuspended in HBSS containing 2% FCS and 2 μg/ml propidium iodide (PI).

Flow cytometric analysis and cell sorting were performed on a dual-laser FACSVantage flow cytometer (Becton Dickinson). Both the Hoechst dye and propidium iodide were excited at 350 nm and their fluorescence was measured at 450 nm and 600 nm. The sorting gate for the hematopoietic stem cell population was established as previously described (see Goodell, M. A. et al., J. Exp. Med., 183:1797-1806 (1996); and Goodell, M. A. et al., International Publication No. WO 9639489 (published Dec. 12, 1996), which references are entirely incorporated herein by reference).

### Bone Marrow and Hematopoietic Stem Cell Transplantation

The female X-linked muscular dystrophic (mdx) recipients were lethally irradiated with 1200 rads given in two doses (600 rads each), 3 hours apart. Bone marrow or hematopoietic stem cells were given intravenously via the tail vein. Mice were maintained on acidified water after transplantation. All animal care was in accordance with institutional guidelines.

### Immunohistochemistry

Detection of dystrophin was performed as previously described (Gussoni, E. et al., Nat. Biotechnol., 14:1012-1016 (1996), the teachings of which are incorporated herein by reference). Briefly, the tibialis anterior (TA) muscle was extracted from the mdx mice, quick frozen in isopentane, and stored at −70° C. 10 μm sections were collected at −20° C. and placed on silanized slides (DAKO). Sections were fixed for 3 minutes in methanol and then transferred to phosphate buffered saline (PBS). Sections were blocked for 20 minutes in PBS containing 10% FCS and 0.05% sodium azide. Dystrophin was detected by incubating the sections with the anti 6-10 antibody (Lidov, H. G. et al., Nature, 348:725-728 (1990)) which recognized the distal rod domain of dystrophin (cDNA residues 6181-9544) for at least 12 hours at 4° C. at 1:500 dilution. After three-10 minute washes with PBS, sections were incubated with a FITC-conjugated anti-rabbit IgG antibody and washed as above.

### Fluorescent In Situ Hybridization

For the simultaneous detection of dystrophin and donor-derived Y chromosomes, in situ hybridization was performed on the same sections as above using a Y chromosome-specific probe (a gift from Dr. Evan Snyder, Children's Hospital, Boston) (see also, e.g., Nishioka, Y., Teratology, 38:181-185 (1988); Harvey, A. R. et al., Brain Res. Mol. Brain Res., 12:339-343 (1992); Prado, V. F. et al., Cytogenet. Cell Genet., 61:87-90 (1992); and Harvey, A. R. et al., Int. J. Dev. Neurosci., 11:569-581 (1993)).

The probe was labeled with digoxigenin-11-dUTP (Boehringer Mannheim) by nick translation as previously described (Gussoni, E. et al., Nat. Biotechnol., 14:1012-1016 (1996), which reference is incorporated herein in its entirety by reference).

Following immunohistochemistry, the sections were fixed in Histochoice (Amresco) for 45 minutes at room temperature, washed in PBS and dehydrated in 50%, 70%, 90%, and 100% ethanol. Sections were denatured for 12 minutes and hybridized with the Y chromosome probe for at least 12 hours at 37° C. in a humidified chamber. After washing and blocking, sections were incubated with a rhodamine-conjugated anti-digoxigenin antibody (Boehringer Mannheim) for 30 minutes. Sections were washed and nuclei were counterstained with 4′-6′ diamidino-2-phenylindole (DAPI) (200ng/ml) diluted in Vectashield mounting medium (Vector Labs). Sections were examined on a Zeiss Axiophot microscope through a triple band-pass filter (Omega). Images of the same microscopic field were taken separately for FITC, DAPI, and rhodamine signals using a CCD camera (Photometrics) and overlaid using the IPLab Spectrum SU2 software (Signal Analytics) on a Macintosh computer.

### EXAMPLE 1

### Immunohistochemical and FISH Analysis of Tibialis Anterior Muscle For Dystrophin Expression and Detection of Y Chromosome-Positive Donor Nuclei After Bone Marrow Transplantation of Lethally Irradiated mdx Mice

To determine whether the transplantation of bone marrow cells carrying a wild type dystrophin gene could restore dystrophin expression within the myofibers of the mdx mouse, nine female mdx mice were lethally irradiated and subsequently injected via the tail vein with 5×10⁵ or 1-5×10⁷ bone marrow cells from male C57BL/10 mice (dystrophin positive). At 5, 8, and 12 weeks after bone marrow transplantation, the tibialis anterior (TA) muscle was analyzed for dystrophin expression by immunohistochemistry, followed by fluorescent in situ hybridization (FISH) using a Y chromosome-specific probe to detect donor-derived (male) cells. Donor nuclei were found in dystrophin-positive myofibers shown at high magnification (100×). Complete reconstitution of the recipient bone marrow by male donor cells was verified by performing FISH with the Y-specific probe on the cells of the bone marrow 5 weeks after bone marrow transplantation.

At 5 weeks after bone marrow transplantation, less than 1% of the myofibers expressed dystrophin (Table 1), similar to background levels. Eight weeks after bone marrow transplantation, however, 1% of the muscle fibers expressed dystrophin in their normal sarcolemmal location, with 25-63% of dystrophin-expressing fibers containing detectable fused donor-derived nuclei (Table 1). By 12 weeks after bone marrow transplantation, as many as 10% of the muscle fibers within an individual mouse expressed dystrophin, with 10-30% of the dystrophin-positive myofibers containing detectable fused Y chromosome-positive nuclei (Table 1).

TABLE 1
Bone marrow transplantation into lethally-irradiated mdx female mice.

| | | | % Dystrophin + | | |
|---|---|---|---|---|---|
| | Age | Number of | myofibers | | % Y + nuclei |
| Animal | (weeks)¹ | Cells Injected | (+ fibers/total)² | | myofibers³ |
| 1 | 5 | 5 × 10⁵ | 0.5 | (5/1122) | 0 |
| 2 | 5 | 5 × 10⁵ | 0.5 | (4/791) | 0 |
| 3 | 5 | 5 × 10⁵ | 1 | (4/474) | 0 |
| 4 | 8 | 5 × 10⁵ | 1 | (11/1053) | 63 |
| 5 | 8 | 5 × 10⁷ | 1 | (11/1252) | 36 |
| 6 | 8 | 5 × 10⁷ | 1 | (4/452) | 25 |
| 7 | 12 | 1 × 10⁷ | 10 | (40/554) | 12 |
| 8 | 12 | 5 × 10⁷ | 5 | (24/456) | 8 |
| 9 | 12 | 5 × 10⁷ | 1 | (6/648) | 33 |

¹Number of weeks after bone marrow transplantation.
²Percent dystrophin-positive fibers determined by counting the number of dystrophin-positive fibers versus the total number of fibers.
³Percentage of dystrophin-positive fibers containing Y chromosome-positive nuclei.

### EXAMPLE 2

### Dystrophin Expression and Detection of Y Chromosome-Positive Nuclei in the Tibialis Anterior Muscle at 12 Weeks After Hematopoietic Stem Cell Transplantation Into Lethally Irradiated Female mdx Recipients

To directly determine whether highly purified hematopoietic stem cells (HSCs) capable of complete reconstitution of lethally-irradiated recipients could give rise to dystrophin-positive myofibers after bone marrow transplantation (BMT), HSCs were purified from normal male C57BL/10 mice as previously described (see Goodell, M. A. et al., J. Exp. Med., 183:1797-1806 (1996); and Goodell, M. A. et al., International Publication No. WO 9639489 (published Dec. 12, 1996), the teachings of which are entirely incorporated herein by reference). These HSCs, termed bone marrow SP cells, have been previously shown to be Sca-1+, lineage marker (B220, Mac-1, Gr-1, CD4, CD5, and CD8) low/negative, and to be capable of the complete engraftment of recipients at very low cell numbers (100-500 cells per mouse) (Goodell, M. A. et al., J. Exp. Med., 183:1797-1806 (1996); Goodell, M. A. et al., Nat. Med., 3:1337-1345 (1997); and Goodell, M. A. et al., International Publication No. WO 9639489 (published Dec. 12, 1996)). Some bone marrow SP cells are also CD34-negative (Goodell, M. A. et al., J. Exp. Med., 183:1797-1806 (1996); Goodell, M. A. et al., Nat. Med., 3:1337-1345 (1997); and Goodell, M. A. et al., International Publication No. WO 9639489 (published December 12, 1996)).

2000-5000 bone marrow SP cells (hematopoietic stem cells) of male origin were injected into the tail veins of 9 lethally-irradiated female mdx mice. As in Example 1, mice were again sacrificed at 5, 8, and 12 weeks after stem cell injection and the TA muscle was analyzed for dystrophin expression by immunohistochemistry and for the presence of male donor cells by FISH using a Y chromosome-specific probe. FISH analysis of the bone marrow confirmed that the female host was completely reconstituted with male donor cells by 5 weeks after stem cell injection.

As was the case with recipients engrafted with unfractionated bone marrow cells, less than 1% of the myofibers expressed dystrophin at 5 weeks (Table 2). At 8 weeks, up to 1% of the muscle fibers expressed dystrophin, with 20-40% of dystrophin-expressing fibers containing fused Y chromosome-positive nuclei (Table 2). At 12 weeks, dystrophin could be detected in up to 4% of the myofibers, with 10-30% of these containing fused donor nuclei (Table 2). In one case, the donor nucleus was found fused further upstream of the dystrophin-positive fiber.

TABLE 2
Hematopoietic stem cell transplantation into lethally-irradiated female
mdx mice.

| | | | % Dystrophin + | | |
|---|---|---|---|---|---|
| | Age | Number of | myofibers | | % Y + nuclei |
| Animal | (weeks)¹ | Cells Injected | (+ fibers/total)² | | myofibers³ |
| 1 | 5 | 2000 | 0.5 | (7/1382) | 0 |
| 2 | 5 | 2000 | 0.5 | (9/1760) | 0 |
| 3 | 5 | 2000 | 0.6 | (2/331) | 0 |
| 4 | 5 | 2000 | 0.7 | (4/591) | 0 |
| 5 | 8 | 2000 | 1 | (18/1600) | 39 |
| 6 | 8 | 2000 | 0.5 | (9/1830) | 22 |
| 7 | 12 | 2000 | 4 | (51/1338) | 12 |
| 8 | 12 | 5000 | 1 | (21/1650) | 28 |
| 9 | 12 | 5000 | 3 | (22/654) | 14 |

¹Number of weeks after hematopoietic stem cell transplantation.
²Percent dystrophin-positive fibers determined by counting the number of dystrophin-positive fibers versus the total number of fibers.
³Percentage of dystrophin-positive fibers containing Y chromosome-positive nuclei.

### EXAMPLE 3

### Position of Fused Donor Nuclei In Dystrophin-Positive Myofibers

In the case of animals transplanted with either unfractionated bone marrow cells or purified SP cells, Y+ nuclei were found to be both centrally and peripherally located within myofibers, but were not observed at the position of satellite cells, the muscle stem cells that normally serve as a local reserve for future myofiber repair (Bischoff, R., in Myology, Engel, A. G. and Franzini-Armstrong, C., Eds, New York: McGraw Hill, pp. 97-119, 1994; and Mauro, A. J., Biophys. Biochem. Cytol., 9:493-495 (1961)) (Table 3).

TABLE 3

| | | | |
|---|---|---|---|
| Satellite¹ | Peripheral² | Central³ | Uncertain⁴ |

Bone marrow transplant (Animals are the same animals as in Table 1)

| | | | | |
|---|---|---|---|---|
| Animal 4 | 0 | 1 | 1 | 4 |
| Animal 5 | 0 | 0 | 3 | 1 |
| Animal 6 | 0 | 0 | 1 | 0 |
| Animal 7 | 0 | 1 | 1 | 2 |
| Animal 8 | 0 | 2 | 0 | 0 |
| Animal 9 | 0 | 0 | 0 | 2 |

Hematopoietic stem cell transplant (Animals are the same animals as in
Table 2)

| | | | | |
|---|---|---|---|---|
| Animal 5 | 0 | 2 | 2 | 0 |
| Animal 6 | 0 | 2 | 0 | 0 |
| Animal 7 | 0 | 1 | 2 | 3 |
| Animal 8 | 0 | 2 | 1 | 3 |
| Animal 9 | 0 | 1 | 0 | 1 |
| TOTAL | 0 | 12 | 11 | 16 |

¹Positioned between the basal lamina and sarcolemma.
²Fused within the myofiber and residing directly below the sarcolemma.
³Fused within the myofiber and located in the center of the myofiber.
⁴Fused within the myofiber and located in the area between the center and peripheral regions.

While these results indicate that bone marrow transplantation in the mdx mouse leads to restoration of dystrophin expression in both regenerating (as indicated by the presence of centrally-located nuclei) and mature muscle fibers (as indicated by the presence of peripherally-located nuclei), they raise the important question of whether bone marrow-derived muscle progenitors actually give rise to satellite cells. Satellite cells normally reside in a quiescent state between the basal lamina and the sarcolemma of the myofiber until they are induced, by muscle damage, to replicate and fuse to regenerate damaged fibers (Bischoff, R., in Myology, Engel, A. G. and Franzini-Armstrong, C., Eds., New York: McGraw Hill, pp. 97-119, 1994); and Mauro, A., J. Biophys. Biochem. Cytol., 9:493-495 (1961)). The repair process induced by bone marrow-derived cells may be distinct from that associated with satellite cells.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method of treating a muscle disease in a mammal in need thereof comprising administering an effective amount of bone marrow cells to the mammal.

2. A method of claim 1 wherein said bone marrow cells are obtained from said mammal to be treated.

3. A method of claim 1 wherein said bone marrow cells are obtained from a donor mammal.

4. The method of claim 1 wherein said mammal is human.

5. The method of claim 4 wherein said muscle disease is a muscular dystrophy.

6. The method of claim 5 wherein said muscular dystrophy is a limb-girdle muscular dystrophy.

7. The method of claim 5 wherein said muscular dystrophy is selected from the group consisting of: Duchenne muscular dystrophy and Becker muscular dystrophy.

8. A method of treating a muscle disease in a mammal in need thereof comprising administering an effective amount of donor bone marrow SP cells to said mammal.

9. A method of claim 8 wherein said bone marrow SP cells are obtained from said mammal to be treated.

10. A method of claim 8 wherein said bone marrow SP cells are obtained from a donor mammal.

11. The method of claim 8 wherein said mammal is human.

12. The method of claim 8 wherein said muscle disease is a muscular dystrophy.

13. The method of claim 12 wherein said muscular dystrophy is a limb-girdle muscular dystrophy.

14. The method of claim 12 wherein said muscular dystrophy is selected from the group consisting of: Duchenne muscular dystrophy and Becker muscular dystrophy.

15. A method of treating a muscle disease in a mammal in need thereof, comprising the steps of:
a) introducing a nucleic acid sequence of interest encoding a desired nucleic acid product into bone marrow SP cells, whereby recombinant bone marrow SP cells are produced; and
b) administering recombinant bone marrow SP cells produced in step a) to said mammal.

16. A method of claim 15 wherein said bone marrow SP cells are obtained from said mammal to be treated.

17. A method of claim 15 wherein said bone marrow SP cells are obtained from a donor mammal.

18. A method of claim 15 wherein said nucleic acid sequence is incorporated into a viral vector.

19. A method of claim 15 wherein said mammal is human.

20. A method of claim 15 wherein said muscle disease is a muscular dystrophy.

21. A method of claim 20 wherein said muscular dystrophy is a limb-girdle muscular dystrophy.

22. A method of claim 20 wherein said muscular dystrophy is selected from the group consisting of: Duchenne muscular dystrophy and Becker muscular dystrophy.

23. A method of claim 22 wherein said desired nucleic acid product is dystrophin.

24. A method for treating a muscular dystrophy in a mammal in need thereof comprising administering an effective amount of bone marrow cells to said mammal.

25. A method of claim 24 wherein said bone marrow cells are obtained from said mammal to be treated.

26. A method of claim 24 wherein said bone marrow cells are obtained from a donor mammal.

27. A method of claim 24 wherein said mammal is human.

28. A method of claim 24 wherein said muscular dystrophy is a limb-girdle muscular dystrophy.

29. A method of claim 24 wherein said muscular dystrophy is selected from the group consisting of: Duchenne muscular dystrophy and Becker muscular dystrophy.

30. A method for treating a muscular dystrophy in a mammal in need thereof comprising administering an effective amount of bone marrow SP cells to said mammal.

31. A method of claim 30 wherein said bone marrow SP cells are obtained from said mammal to be treated.

32. A method of claim 30 wherein said bone marrow SP cells are obtained from a donor mammal.

33. A method of claim 30 wherein said mammal is human.

34. A method of claim 30 wherein said muscular dystrophy is a limb-girdle muscular dystrophy.

35. A method of claim 30 wherein said muscular dystrophy is selected from the group consisting of: Duchenne muscular dystrophy and Becker muscular dystrophy.

36. A method of treating a muscular dystrophy in a mammal in need thereof, comprising the steps of:
a) introducing a nucleic acid sequence of interest encoding a desired nucleic acid product into bone marrow SP cells, whereby recombinant bone marrow SP cells are produced; and
b) administering said recombinant bone marrow SP cells produced in step a) to said mammal.

37. A method of claim 36 wherein said bone marrow SP cells are obtained from said mammal to be treated.

38. A method of claim 36 wherein said bone marrow SP cells are obtained from a donor mammal.

39. A method of claim 36 wherein said mammal is human.

40. A method of claim 36 wherein said nucleic acid sequence is incorporated into a viral vector.

41. A method of claim 36 wherein said muscular dystrophy is a limb-girdle muscular dystrophy.

42. A method of claim 36 wherein the muscular dystrophy is selected from the group consisting of: Duchenne muscular dystrophy and Becker muscular dystrophy.

43. A method of claim 42 wherein said desired nucleic acid product is dystrophin.
